(19) United States
(12) Patent Application Publication
(10) Pub. No.: US 2003/0039678 A1
(43) Pub. Date: Feb. 27, 2003

(54) Xenograft bone matrix for orthopedic applications

(76) Inventors: Kevin R. Stone, Mill Valley, CA (US); Thomas J. Turek, San Francisco, CA (US)

Correspondence Address: McDERMOTT, WILL & EMERY, 28 State Street, Boston, MA, 02109-1775, US

(21) Appl. No.: 10/103,613

(22) Filed: Mar. 21, 2002

Related U.S. Application Data
(63) Continuation-in-part of application No. 09/585,509, filed on Jun. 1, 2000, now Pat. No. 6,383,732.
(63) Continuation-in-part of application No. 09/646,376, filed on Sep. 14, 2000.
Application No. 09/646,376 is a 371 of international application No. PCT/US99/05661, filed on Mar. 15, 1999.
(63) Continuation-in-part of application No. 09/647,726, filed on Dec. 4, 2000.
Application No. 09/647,726 is a 371 of international application No. PCT/US99/05646, filed on Mar. 15, 1999.
(60) Provisional application No. 60/078,139, filed on Mar. 16, 1998.
(60) Provisional application No. 60/100,755, filed on Sep. 17, 1998.
(60) Provisional application No. 60/080,491, filed on Apr. 2, 1998.
(60) Provisional application No. 60/278,192, filed on Mar. 23, 2001.

(51) Int. Cl. A61K 35/32; C12N 5/08

(52) U.S. Cl. 424/424; 435/366; 424/549

(57) ABSTRACT

The invention provides for the use of an improved xenograft bone particulate with respect to osteo-integration and bone remodeling, while diminishing the primate-to-pig immunological response using established bone-processing technique. Work was carried out using undecalcified bone to determine immunocompatibilty and bone remodeling potential of processed porcine bone struts following onlay graft implantation. New bone formation was evident, including the infiltration of cellular materials responsible for fusion and bone reconstruction.

### CLAIM OF PRIORITY

This application is a continuation-in-part of U.S. patent application Ser. No. 09/585,509, filed Jun. 1, 2000, now allowed, which claims priority to U.S. patent application Ser. No. 09/248,476, filed Feb. 11, 1999, now U.S. Pat. No. 6,231,608, which issued on May 15, 2001. This patent application is also a continuation-in-part of U.S. patent application Ser. No. 09/646,376, filed Sep. 14, 2000, which is the U.S. National Stage of Int'l Patent Application No. PCT/US99/05661, filed Mar. 15, 1999, published as WO 99/47080 on Sep. 23, 1999, and which claims priority to U.S. Provisional Patent Applications Serial No. 60/078,139, filed Mar. 16, 1998, and Ser. No. 60/100,755, filed Sep. 17, 1998. This patent application is also a continuation-in-part of U.S. patent application Ser. No. 09/647,726, filed Dec. 4, 2000, which is the U.S. National Stage of Int'l Patent Application No. PCT/US99/05646, filed Mar. 15, 1999, published as WO 99/51170 on Oct. 14, 1999, and which claims priority to U.S. Provisional Patent Application Ser. No. 60/080,491, filed Apr. 2, 1998. This application further claims priority to U.S. Provisional Patent Application Ser. No. 60/278,192, filed Mar. 23, 2001.

### FIELD OF THE INVENTION

The invention relates to the treatment of defective bone, and in particular, to replacement and repair of defective or damaged bone using a substantially immunologically compatible bone matrix from a non-human animal.

### BACKGROUND OF THE INVENTION

Autogenous bone grafting has long been established as the treatment of choice for management of skeletal defects. It is estimated that United States surgeons perform over 400,000 procedures requiring bone grafting each year. (Lane et al., Orthopedic Special Edition 6(1): 61-64 (2000), Piper-Jaffray, Orthopedics Overview (1999)) These grafts are used in spinal fusion, fracture non-union, total joint revision and maxillofacial reconstruction procedures. Problems with autogenous bone harvest from the iliac crest site are donor site morbidity and limitations on the overall volume of graft material available (Seiler & Johnson, J. South. Orthop. Assoc. Summer: 9(2): 91-7 (2000), Boden et al., Spine 20: 412-420 (1995)).

A variety of natural and synthetic bone graft substitutes or extenders have been developed, falling into three general categories: (a) Synthetics, (b) Bioceramics and (c) Bio-Derived (Kenley et al., Pharm. Res. 10(10): 1393-401 (Oct. 1993), Sigurdsson et al., Int. J. Periodontics Restorative Dent. 16(6): 524-37 (Dec. 1996), Lane et al., Orthopedic Special Edition 6(1): 61-64 (2000)). Bio-derived bone graft substitutes range from purified collagen scaffolds to allograft and xenograft mineralized and demineralized matrix materials. Allograft are currently used in the majority of non-autogenous grafting procedures and have achieved the best clinical results to date due to inherent osteoconductivity, process determined osteoinductivity and biomaterial compatibility (Bauer & Muschler, Clin. Orthop. 371: 10-27 (Feb. 2000), Goldberg, Clin. Orthop. (381): 68-76 (Dec. 2000)). Cadaver derived materials have focused on mineralized and stress bearing constructs in machined struts or dowels for onlay/augmentation procedures and demineralized particulate formulations optimizing surgical placement and speed of osteo-integration for defect and void repair.

The major immunological obstacle for the use of pig tissues as implants in humans is the natural anti-Gal antibody, which comprises 1% of antibodies in humans and monkeys and which binds to α-Gal epitopes (Galα1-3Gal β1-4GlcNAc-R) expressed on pig glycoproteins. CrossCart Inc. has developed a method for eliminating α-Gal epitopes by the use of recombinant α-galactosidase. This enzyme destroys the α-Gal epitope by cleaving the terminal galactosyl unit. Galactose is released following the cleavage of Galα1-3Gal β1-4GlcNAc-R to Galβ1-4GlcNAc-R+Gal.

Previous studies have clearly demonstrated the immunogenic contribution of the ∞-gal epitope on pig to primate/human grafting and have devised a method to eliminate this response using the ∞-galactosidase enzyme (Galili & Andrews, J. Human Evolution 29:433 (1995), Galili et al., Transplantation 65:1129 (1998)). These studies include ∞-galactosidase treatment of porcine articular and fibro-cartilage connective tissues and evaluation in a primate model (Galili et al., Transplantation 63: 646 (1997)). ∞-Gal epitope is primarily responsible for pig to primate/human xenograft rejection and demonstrate that rejection can be overcome in non-viable connective tissue of pig origin by enzymatic irreversible destruction of the ∞-Gal epitope with the recombinant enzyme ∞-galactosidase produced in yeast.

Considering the limited supply of cadaveric bone and potential for disease transmission, there is a need in the art to further the understanding of the osteoconductive property of xenograft bone grafting materials, the osteoinductive potential of porcine bone resulting from endogenous growth factors, and specific immunocompatibility of pig to primate bone grafting with the ultimate aim of achieving pig to human compatibility (Aichelmann-Reidy & Yukna, Dent Clin North Am Jul; 42(3): 491-503 (1998)).

### SUMMARY OF THE INVENTION

This invention provides an effective xenograft demineralized and deantigenated bone matrix, with osteoconductive and osteoinductive potential following treatment with ∞-galactosidase to eliminate ∞-Gal epitopes. This bone matrix has an increased immunocompatibility. The invention also provides a treatment strategy for xenograft bone particulate and shows the osteoconductive and osteoinductive properties while diminishing the human to pig immunological response. Established bone and novel xenograft processing techniques are used with proven assessment tools and animals model. The invention is useful for facilitating the use of demineralized and deantigenated porcine bone matrix as a bone graft for defects of the skeletal system. This source of bone grafts material provides surgeons an alternative to autografts, allografts, and synthetic grafts in clinical use.

In several embodiments, the invention uses treated cortical struts for immunological profile and demineralized porcine bone processing in a rat cranial defect model to assess the osteoconductive, osteoinductive and biocompatibility properties through radiography and histology.

### BRIEF DESCRIPTION OF THE DRAWINGS

FIG. 1 is a set of photomicrographs of undecalcified histological sections from untreated (freezing only) cortical strut grafted sites at 26 weeks post-operatively. Micrograph notation: (F)=femur, G=xenograft strut, N=new bone. FIG. 1A is the left femur. FIG. 1B is the right femur. (1×magnification, basic fuchsin staining).

FIG. 2 is a set of photomicrographs of undecalcified histological sections from two treated (α-galactosidase+glutaraldehyde) cortical strut grafted sites at 26 weeks post-operatively. (1×magnification, basic fuchsin staining).

### DETAILED DESCRIPTION

The efficacy of a xenograft demineralized and deantigenated bone matrix is here assessed with respect to osteoconductive, potential osteoinductive and immunological properties and characteristics. Initial assessment of immunology uses ∞-galactosidase treated porcine cortical struts in a primate femoral onlay study. Final assessment uses decalcified bone particulate that has been treated with ∞-galactosidase to eliminate ∞-Gal epitopes. The biocompatibility, osteoconductive and osteoinductive potential of the demineralized matrix are assessed in a rat cranial defect model using radiography and histology.

The overall unifying concept of the invention is that processed xenogeneic porcine demineralized bone treated with ∞-galactosidase is osteoconductive, osteoinductive and immunocompatible. The xenogeneic porcine demineralized bone treated with ∞-galactosidase is also biocompatible, porous, resorbable, and space maintaining.

The term “xenograft” is synonymous with the term “heterograft” and refers to a graft transferred from an animal of one species to one of another species. Stedman's Medical Dictionary, Williams & Wilkins, Baltimore, Md. (1995). The term “xenogeneic”, as in, for example, xenogeneic soft tissue refers to soft tissue transferred from an animal of one species to one of another species. Id. Once implanted in an individual, a xenograft provokes immunogenic reactions such as chronic and hyperacute rejection of the xenograft. The term “chronic rejection”, as used herein refers to an immunological reaction in an individual against a xenograft being implanted into the individual. Typically, chronic rejection is mediated by the interaction of IgG natural antibodies in the serum of the individual receiving the xenograft and carbohydrate moieties expressed on cells, and/or cellular matrices and/or extracellular components of the xenograft. For example, transplantation of cartilage xenografts from non-primate mammals (e.g., porcine or bovine origin) into humans is primarily prevented by the interaction between the IgG natural anti-Gal antibody present in the serum of humans with the carbohydrate structure Galα1-3Galβ1-4GlcNAc-R (α-galactosyl or α-gal epitope) expressed in the xenograft. K. R. Stone et al., Porcine and bovine cartilage transplants in cynomolgus monkey: I. A modelfor chronic xenograft rejection, 63 Transplantation 640-645 (1997); U. Galili et al., Porcine and bovine cartilage transplants in cynomolgus monkey: II. Changes in anti-Gal response during chronic rejection, 63 Transplantation 646-651 (1997). In chronic rejection, the immune system typically responds within one to two weeks of implantation of the xenograft. In contrast with “chronic rejection”, “hyper acute rejection” as used herein, refers to the immunological reaction in an individual against a xenograft being implanted into the individual, where the rejection is typically mediated by the interaction of IgM natural antibodies in the serum of the individual receiving the xenograft and carbohydrate moieties expressed on cells. This interaction activates the complement system causing lysis of the vascular bed and stoppage of blood flow in the receiving individual within minutes to two to three hours.

The following EXAMPLES are presented in order to more fully illustrate the preferred embodiments of the invention. These EXAMPLES should in no way be construed as limiting the scope of the invention, as defined solely by the appended claims.

### EXAMPLE I

Evaluation of Xenograft Materials in Primates—Cancellous and Cortical Bone Models

In this EXAMPLE, methods have been developed for decreasing the immune response against porcine tissue implanted in monkeys, by eliminating the α-Gal epitopes (Galα1-3Galβ1-4GlcNAc-R) with recombinant α-galactosidase, and mild glutaraldehyde fixation. Results using non-decalcified bone struts provide supporting evidence for the use of bone particulates for bone repair.

Background. Previously, CrossCart Inc. (San Francisco, Calif., USA) has extensively characterized a porcine bone patellar tendon bone anterior cruciate ligament (ACL) reconstruction device. This composite device consists of a sterile and biocompatible collagen tendon with cortical/cancellous bone plugs on each end. An irradiation-processing step significantly reduces viral agents from spiked samples. In previous primate bone testing, CrossCart used porcine bone grafts in the femur of primates to evaluate solid bony fusion to screen process variables for new bone formation and fusion of implants with host tissue.

Methods: In this EXAMPLE, eighteen adult male rhesus monkeys weighing 9-18.5 kg were used to characterize cortical bone healing and graft incorporation. The anesthetized monkeys received bilateral cortical on-lay strut grafts that were secured to the mid-femur held in place with proximal and distal wires, and cancellous defects, 8 mm in diameter by 8 mm deep were created in the distal femur and proximal tibia. The screening groups consisted of xenograft cortical struts (TABLE I) or cancellous bone (TABLE II) material treated as follows: (a) freeze only (b) alcohol+freeze (c) α-galactosidase+gluteraldehyde and α-galactosidase+gluteraldehyde+hydrogen peroxide.

Twelve animals received two bilateral on-lay xenograft strut grafts approximately 5 cm in length and 0.5 cm wide on the lateral and posterior surfaces of the femur. Cortical bone healing was evaluated at 6 and 26 weeks post-implantation. Cancellous bone healing with xenograft cancellous cylindrical plugs was evaluated in bilateral defects created in the metaphyseal region of the distal femur and/or proximal tibia.

A total of 36 cancellous bone defects in 14 animals were evaluated with the addition of xenograft plugs at 6, 12, and 26 weeks post-implantation. Eight control (empty) cancellous defects were evaluated in the distal femurs of eight animals at 26 weeks post-implantation. Six animals were necropsied at 6 weeks, one at 12 weeks, and 11 at 26 weeks post-implantation. Plain film radiographs were taken at intervals to test the progression of healing of the femurs and tibias. All sections were then histologically examined by preparing undecalcified histological sections to determine tissue response, residual implant material, quality and amount of new bone formation, graft incorporation and remodeling.

Each cortical strut and cancellous defect site was observed for gross appearance. The cortical strut grafts were manually determined to be stable or unstable prior to removing the wires. If a strut was very unstable, the wires were left in place. Presence of fibrous tissue and degree of bone contact between the strut graft and femur cortex was noted. The length, width and height in millimeters of each strut graft were measured and noted. Visual observation of the overall incorporation and remodeling of the strut graft was made and recorded as well as any other notable findings related to the gross appearance. Similarly, the cancellous defect sites were observed for the presence of graft material, fibrous tissue, incorporation with the host bone, and visual changes in or around the defect. Two struts were placed on each femur. A summary of implanted graft materials for cortical strut on-lay graft model by treatment type is depicted in TABLE I below.

TABLE I

| Animal | | Number | | |
|---|---|---|---|---|
| Number | Location | of sites | Treatment type | Duration |
| D831 | Bilateral femur | 4 | Untreated (freezing) | 26 weeks |
| L551 | Bilateral femur | 4 | Limited treatment (alcohol + freezing | 26 weeks |
| M338 | Bilateral femur | 4 | Treated (α Gal + gluteraldehyde) | 6 weeks |
| M556 | Bilateral femur | 4 | Treated (α Gal + gluteraldehyde) | 6 weeks |
| M002 | Bilateral femur | 4 | Treated (α Gal + gluteraldehyde) | 6 weeks |
| J761 | Bilateral femur | 4 | Treated (α Gal + gluteraldehyde) | 26 weeks |
| N049 | Bilateral femur | 4 | Treated (α Gal + gluteraldehyde) | 26 weeks |
| G185 | Bilateral femur | 4 | Treated (α Gal + gluteraldehyde) | 26 weeks |
| J427 | Bilateral femur | 4 | Treated (α Gal + gluteraldehyde) | 26 weeks |
| J730 | Bilateral femur | 4 | Treated (α Gal + gluteraldehyde) | 26 weeks |
| J843 | Bilateral femur | 4 | Treated (α Gal + gluteraldehyde) | 26 weeks |
| J980 | Bilateral femur | 4 | Treated (α Gal + gluteraldehyde + H₂O₂) | 26 weeks |

A summary of implanted graft materials for cancellous bone defect graft model by treatment type is shown in TABLE II below. One cylindrical plug graft was placed per defect site.

TABLE II

| Animal | | Number | | |
|---|---|---|---|---|
| Number | Location | of sites | Treatment type | Duration |
| D831 | Bilateral femur | 2 | Untreated (freezing) | 26 weeks |
| L551 | Bilateral femur | 2 | Limited treatment (alcohol + freezing | 26 weeks |
| J849 | Bilateral femur | 4 | Treated (α Gal + gluteraldehyde) | 6 weeks |
| | Bilateral tibia | | | |
| J625 | Bilateral femur | 4 | Treated (α Gal + gluteraldehyde) | 6 weeks |
| | Bilateral tibia | | | |
| L943 | Bilateral femur | 4 | Treated (α Gal + gluteraldehyde) | 6 weeks |
| | Bilateral tibia | | | |
| N140 | Right femur | 3 | Treated (α Gal + gluteraldehyde) | 26 weeks |
| | Bilateral tibia | | | |
| M889 | Right femur | 3 | Treated (α Gal + gluteraldehyde) | 26 weeks |
| | Bilateral tibia | | | |
| J761 | Right femur | 3 | Treated (α Gal + gluteraldehyde) | 26 weeks |
| | Bilateral tibia | | | |
| N049 | Right femur | 3 | Treated (α Gal + gluteraldehyde) | 26 weeks |
| | Bilateral tibia | | | |
| G185 | Right femur | 1 | Treated (a Gal +gluteraldehyde) | 26 weeks |
| J427 | Right femur | 1 | Treated (a Gal +gluteraldehyde) | 26 weeks |
| J730 | Right femur | 1 | Treated (a Gal +gluteraldehyde) | 26 weeks |
| J843 | Right femur | 1 | Treated (a Gal +gluteraldehyde) | 26 weeks |
| D145 | Bilateral femur | 4 | Treated (a Gal +gluteraldehyde+H₂O₂) | 12 weeks |
| | Bilateral tibia | | | |

No animals experienced adverse clinical reaction related to the implanted materials or surgical procedures. All animals were fully weight bearing by the end of the second post-operative week. The in vivo analysis included the administration of oxytetracycline hydrochloride (20 mg/kg body weight) and fluorochrome at 14 and 7 days prior to the scheduled necropsy. Bilateral antero-posterior and lateral radiographs of the lower limbs were obtained immediate post-operative, at 3 months and at necropsy. All radiographs were taken within three days of the scheduled radiograph date. Blood samples were intermittently taken for anti-Gal activity.

Results: The treated xenograft material displayed improved biological performance when implanted into the non-human primate model. Only the results of the 26-week test are presented here. Specifically the α-galactosidase and gluteraldehyde treatment of porcine and cortical and cancellous bone grafts demonstrated less inflammatory reaction as compared to untreated xenograft cortical and cancellous bone graft controls. Furthermore, this test group also showed increased remodeling, graft incorporation and new bone formation in the in the cortical strut graft compared to untreated controls. The cancellous plug grafts placed in the distal femur and proximal tibia similarly showed increased graft incorporation and remodeling compared to untreated xenograft controls. The data is shown in the TABLE III below represents the summed response for both axial and longitudinal bone.

TABLE III

| Cortical Bone | n | Control | n | Freeze | n | Peroxide | n | α-Gal |
|---|---|---|---|---|---|---|---|---|
| Remodeling | 5 | 5/5 | 5 | 0/5 | 8 | 3/8 | 33 | 17/33 |
| Graft Incorporation | 5 | 10 | 5 | 33.4 | 8 | 40.1 | 33 | 44.5 |
| Inflammation | 5 | 1.8 | 5 | 1.35 | 8 | 1.0 | 33 | 0.85 |
| New Bone Formation (%) | 5 | 1.2 | 5 | 1.5 | 8 | 1.5 | 33 | 1.8 |

The histological data of cancellous bone defects is shown in TABLE IV below:

TABLE IV

| Cancellous Bone | n | Control | Freeze | n | Empty Defect | n | Peroxide* | n | α-Gal |
|---|---|---|---|---|---|---|---|---|---|
| Remodeling | 2 | 0/2 | ½ | 7 | — | 4 | 0/4 | 15 | 7/15 |
| Graft Incorporation | 2 | 10 | 57.5 | — | — | 4 | 11.3 | 15 | 53.2 |
| Inflammation | 2 | 2 | 0.8 | 7 | 0 | 4 | 0.5 | 15 | 0.8 |
| New Bone Formation (%) | 2 | 57.5 | 60 | 7 | 75 | 4 | 8.0 | 15 | 41.3 |

*Necropsied at 12 weeks

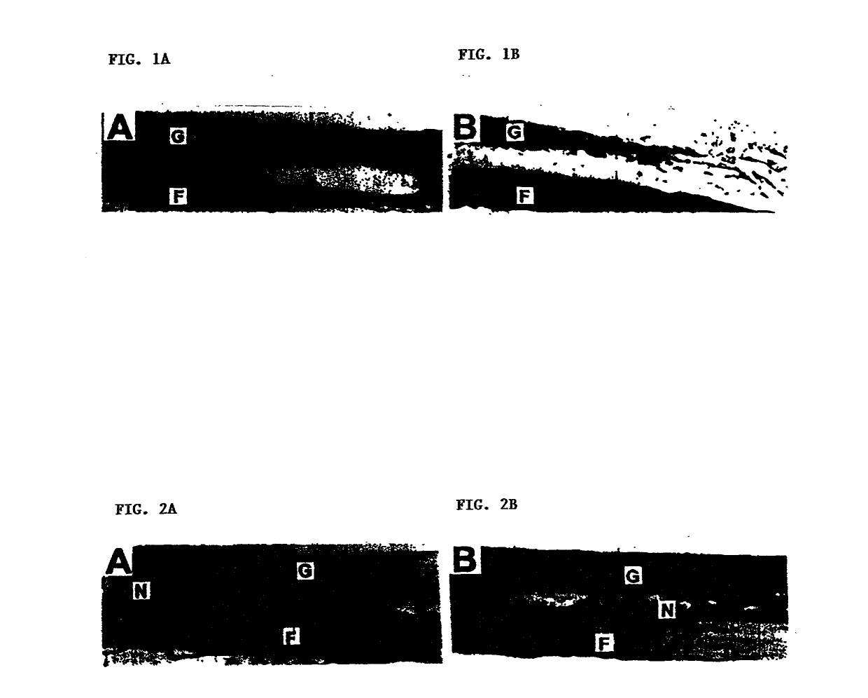

Histological Analysis: The photomicrographs of undecalcified histological sections from untreated (freezing only) cortical strut grafted sites at 26 weeks post-operatively are shown in FIG. 1A and FIG. 1B. In FIG. 1A, graft incorporation was approximately 20% to 30% on the left femur. Mineralizing cartilage is observed between the graft and host bone. Residual graft is shown at the top of figure and the femur cortex is shown at the bottom (1×magnification, basic fuchsin). Graft incorporation on the right femur (FIG. 1B) was considerably lower due to fibrous tissue interposition and a significant gap. Note the resorption of graft distally to the right of the image. The micrographs are at 1×magnification, stained using basic fuchsin.

The photomicrographs of undecalcified histological sections from two treated (α-galactosidase+glutaraldehyde) cortical strut grafted sites at 26 weeks post-operatively are shown in FIG. 2A and FIG. 2B. In FIG. 2A, graft incorporation was approximately 55% in this site. New bone is seen bridging from the femur cortex to the residual graft. In FIG. 2B, significant contact between host femur cortex, new bone bridge and strut graft was observed. The micrographs are at 1×magnification, stained using basic fachsin.

Discussion: The (α-galactosidase and gluteraldehyde treated cortical strut grafted sites exhibited minimal graft resorption, limited new bone formation and no inflammatory response at 6 weeks post-operatively. At 26 weeks post-operative, there was minimal graft resorption with a significant amount of new bone formation and bony incorporation along the host cortex bridging to the graft. Some fibrous tissue was present in the gap interfaces between strut and host cortex and the inflammatory reaction minimal in all cases. The inflammatory reaction to the untreated grafts was moderate to severe characterized by osteoclastic graft resorption and the presence of foreign body giant cells in the surrounding tissues. The results of all inflammatory reactions are shown in TABLE III and TABLE IV, above.

Histological analysis of cancellous plug grafted sites evaluated at 6 weeks post-operatively showed very early and limited new bone formation. Graft incorporation was related to the degree of graft resorption that was mild to moderate in the majority of defects. The inflammatory reaction to the treated cancellous grafts at 6 weeks was none to mild in the majority of sites. At 26 weeks, the amount of new bone formation was greater for the treated cancellous plug grafted sites as compared to those evaluated at 6 weeks. In the α-galactosidase+gluteraldehyde group, graft incorporation was higher with a corresponding increase in graft resorption and a lower percentage of residual graft. The majority of cancellous graft sites had none to mild inflammatory response.

Conclusion: The results of this EXAMPLE support previous findings in which recombinant α-galactosidase treatment of porcine patellar tendons resulted in a significant reduction in Anti-Gal humoral response and limited cellular infiltration (Galili, Science and Medicine, 32 (Sept./Oct. 1998). This EXAMPLE shows that bone grafts can be similarly treated with α-galactosidase to deter the inflammatory response and promote graft incorporation. Although this EXAMPLE I does not specifically address osseus union of bone fractures, the model is, however, directly applicable to bone repair mechanisms where the union of bone is anticipated and where the infiltration of cellular materials responsible for fusion and bone reconstruction are actively recruited.

### EXAMPLE II

Xenograft Bone Matrix for Orthopedic Applications

This EXAMPLE refines the treatment regimen of EXAMPLE I to obtain maximum benefit in removal of α-Gal epitopes from xeno-active tissues and promote accelerated osseus union.

Process Development. Diaphyseal bone is harvested from 6 to 12 month old swine from a medical grade abattoir that also supplies porcine aortic heart valves for human implantation. After dissection of soft tissue, manual periosteal stripping and marrow removal, bone pieces are subjected to consecutive hypertonic, hypotonic and alcohol rinses. The bone is then milled to sieve standardized 150 to 500 μm particle size (Zhang et al., J. Periodontol. 68(11): 1085-92 (1997)). After sizing, the particles are subjected to consecutive hydrogen peroxide and alcohol washes. Downstream processing includes separate hydrochloric acid decalcification and enz;yatic treatment. Protocols have been established to characterize the α-galactosidase enzyme, as described below:

Assay For α-Galactosidase. The enzyme (α-galactosidase (previously cloned from coffee beans and genetically expressed in the yeast Pichia pastoris) has been well-characterized (Zhu et al., Arch. Biochem. Biophysics 324: 65 (1995)). α-galactosidase is an exoglycosidase of molecular weight 41 kDa that is diffusely distributed in nature. It functions by cleaving the terminal α-galactose residue from oligosaccharide chains from cells. The activity of recombinant enzyme is determined by reacting diluted enzyme with p-nitrophenyl-α-galactoside substrate, for 10 minutes at room temperature (Zhu et al., Arch. Biochem. Biophysics 827:324 (1996)). The absorbance of p-nitrophenol in each solution is read at 405 mn. The enzyme is stable at 37° C., 24° C., and 4° C. and is affected by repeated freezing and thawing. The activity of each batch of enzyme is checked prior to use in assays.

Determination of α-Gal Epitope Expression. An “ELISA inhibition” assay was developed for the determination of α-gal epitope expression on various tissues. This assay is a modification of a radioimmunoassay solid-phase method, previously developed to measure mammalian glycoproteins. The interaction of M86 anti-Gal antibody with α-gal epitopes on cells is measured by the activity of free M86 remaining in the supernatant after incubation with α-gal-BSA (solid-phase). With minor modifications, the assay can be used for the determination of α-Gal epitope expression on bone particulate homogenates. Demineralized bone particulates are incubated at various concentrations with the monoclonal anti-Gal antibody designated M86 at a dilution of 1:100 of the antibody. After overnight incubation with constant rotation the particles and bound antibody is removed by centrifugation. The remaining anti-Gal antibody in the supernatant are determined by ELISA with α-Gal epitope linked to BSA (α-Gal BSA) as solid phase antigen. There is a direct correlation between the number of α-Gal epitopes expressed in the bone particles and the binding of the monoclonal anti-Gal antibody to these particles (i.e. removal of the antibody from the supernatant). Bone particulates devoid of α-Gal epitopes bind no anti-Gal and thus does not decrease the subsequent binding of the antibody to α-Gal BSA as a result of overnight incubation with the antibody.

Determination ofEnzyme Protein Concentration—Specific Activity Determination. This assay employs the Sigma Diagnostics Microprotein-PR™ kit that quantitatively determines the amount of protein in solution. The reaction medium consists of 0.05 mmol/L pyrogallol red, 0.16 mmol/L sodium molybdate. The protein standard solution consists of human albumin (50 mg/100 ml) in saline with 0.1% sodium azide as a preservative. 95 μl of the pyrogallol reagent is added into each well. Deionized water is used as a blank. Into the test wells are added 5 μl of enzyme solution (1/50 dilution). The standard albumin solution is added into separate wells. The multiwell plate and contents is incubated for 3 minutes at 37° C. The absorbance is determined at 600 nm. The protein concentration is calculated using the formula: Protein (mg/dl)=Aₜₑₛₜ−A_{blank}/A_{standard}−A_{blank}X Concentration of Standard.

Procedurefor Epitope Determination in Bone Particulates. This assay is a modification of a radioimmunoassay solid-phase method, previously developed to measure mammalian glycoproteins. The interaction of M86 anti-Gal antibody with (α-gal epitopes on cells is measured by the activity of free M86 remaining in the supernatant after incubation with α-gal-BSA. Bone particulates are subjected to vigorous homogenization in PBS pH 7.2/3. The final concentrate is then diluted to a concentration of approximately 200 mg/ml and then serially diluted with PBS containing 1% BSA. Each diluted sample (0.1 ml) is then pipetted into a microcentrifuge tube. The monoclonal anti-Gal antibody (M86), at a dilution of 1:50, is then also added to each tube in 0.1 ml aliquots. A final dilution of 1:100 of M86 antibody subsequently provides a 50% maximum binding to α-gal-BSA. This dilution is suitably sensitive for determining anti-Gal antibody binding to epitopes. The tubes containing the homogenate and monoclonal antibody are then maintained at 4° C. with continuous rotation overnight. During this period the M86 antibodies begin the binding process to the α-gal epitopes in particles of the homogenate suspension. Finally, the tissue fragments that bind to antibody molecules are removed by centrifugation in an Eppendorf microfuge tube at 14,000 rpm (35,000×g). Hence, ELISA results determine the activity of the M86 antibody remaining in the supernatant with α-gal-BSA as the solid-phase antigen and horseradish peroxidase-conjugated goat anti-mouse IgM second antibody (IgM-HRP; Axcell Laboratories). Color development are generated by the addition of o-phenylenediamine (OPD) at a concentration of 1 mg/ml in peroxide buffer, pH 5.5, containing 10 μl/ml of 30% hydrogen peroxide. Since particles containing α-Gal epitopes remove the antibody prior to the ELISA procedure, the interaction results in “inhibition” of the subsequent M86 binding to the solid-phase α-Gal-BSA. Comparison of the inhibition curves of the test homogenate M86 level with those of a standard value obtained from the M86 antibody level prior to α-Gal treatment provide data that quantifies the apparent increase in antibody titer. Thus, the concentration of a-galactosidase that results in complete elimination of α-Gal epitopes is determined by observing no binding of M86 to the particles.

Procedure for Determination of Anti-Bone Matrix Antibodies in Sera. Antibody production to bone matrix proteins is determined by ELISA with particulate bone matrix as solid phase antigen. The particles are homogenized to a size of 1-10 μm and dried on ELISA plates as 100 ug/well. Hence, the procedure originally used for cartilage and ligaments is applied to bone in this test. An ELISA test is performed using either untreated porcine bone particulates or α-galactosidase-treated bone particulates samples plated, dried and blocked. Dilutions of serum, starting at 1:50, in 50 μl amounts are then added to the wells. The plates are kept for 2 hr at room temperature, washed 4 times with PBS-Tween and reacted with anti-human IgG-HRP (Dako) diluted 1:1000 for 1 hr at room temperature. After 5 further washes with PBS-Tween, a color develops when incubated with OPD for a reaction time of 3 to 5 minutes. ELISA absorbance values are compared in samples of sera collected pre- and post-implantation from each animal. A stable value or increase in antibody titer provides a measure about the anti-bone immune response.

Determination of α-Galactosidase Content in Bone Particles. Bone particulates are weighed, then dissolved in a fixed volume of PBS (pH 7.0) plus 0.1% Triton X100 and homogenized. The homogenate are stored at 32° C. for 30 minutes followed by 10 minutes of centrifugation at 12000 g. The supernatant is decanted and Millipore filtered. The α-galactosidase activities are determined in the supernatant. A similar extraction procedure is conducted in bone particulates immediately post α-galactosidase treatment. These data provide information pertaining to the precise concentration of residual α-galactosidase remaining in the tissue following processing. Spiking an additional homogenates with a known amount of α-galactosidase and similarly determining the activity in the extract validates the assay.

Summary ofProtocols. Enzyme optimization is conducted in groups, as described in TABLE V.

TABLE V
Enzyme Optimization and Process Development

| Enzyme Optimization | | | Enzymatic Treatment |
|---|---|---|---|
| Group | Treatment 1 | Treatment 2 | Level |
| A | decalcification | ∝-galactosidase | 90 U/gm |
| B | decalcification | ∝-galactosidase | 30 U/gm |
| C | decalcification | ∝-galactosidase | 10 U/gm |
| D | decalcification | ∝-galactosidase | 3 U/gm |
| E | decalcification | buffer | 0 |

Porcine graft materials treated with α-galactosidase enzyme (100 units/ml) have been successfully deantigenated using a specified enzyme to gram of tissue ratio (Galili et al., Transplantation 65:1129 (1998); Galili et al., Transplantation 63; 646 (1997)). Based on previous experience with cartilage, the enzyme should penetrate into the decalcified bone granules and destroy the ∞-Gal epitopes in the bone matrix. The elimination of the ∞-Gal epitopes is measured at various ∞-galactosidase concentrations by the ELISA inhibition assay with a monoclonal antibody to ∞-Gal epitopes as we previously described (Galili et al., Transplantation 65:1129 (1998))

Although the effective surface area of processed connective tissues has not been measured, the effective surface area of milled bone particulate (150-500 um range) is many orders of magnitude greater. Particulate processing provides vast surface area and minimal diffusional path-length, maximizing epitope presentation and resultant enzyme/product clearance. Other specifics for process development include scaleable process design, implementing scaleable reactors from cell culture technology. Final processing of prepared matrix materials includes lyophilization, vialing and terminal sterilization using 2.5 MRAD ionizing radiation. Once the optimization and processing has been standardized, materials for in-vivo testing are prepared.

Rat Cranial Defect Model Test System. The rat cranial defect model has been established as a screening assay for osteoconductive and osteoinductive properties of bone grafting materials (Hollinger & Kleinschmidt, J. Craniofac. Surg. 1(1): 60-8 (Jan. 1990); Hollinger et al., Clin. Orthop. (267): 255-63 (Jun. 1991)). The Long Evans rats are quarantined for one-week prior to use. The rats are placed in a bell jar and subjected to inhalant anesthesia (isofluorane). Once sedated, the rats are transferred to a sterile operating field and prepared for surgery. The animals are then injected with ketamine/xylazine cocktail (100 mg/20 mg) as an initial induction dosage followed by a maintenance dose of 50 mg/10 mg cocktail as required. The breathing depth is monitored and the toe pinch reflex applied to evaluate the depth of anesthesia. Ophthalmic ointment is applied to the eyes to prevent dehydration.

The rat cranial defect model in this EXAMPLE uses Long Evans rats, in which an 8 mm trephine defect is created in the cranium (Hollinger et al., Surgery 107(1): 50-4 (Jan. 1990)). Animals are skeletally mature with adult rats weighing between 250-300 gm. Rat model details include a four-week assessment time point with six animals per test group (Hollinger et al., Clin. Orthop. (267): 255-63 (Jun. 1991); Schmitz et al., Acta Anat (Basel) 138(3): 185-92 (1990)). After the surgical site is prepared using consecutive applications of betadine and 70% isopropyl alcohol, a linear incision is made from the nasal bone to mid-sagittal crest. Soft tissues are reflected and the periosteum dissected from the exposed occipital, frontal and parietal bones. An 8 mm craniotomy defect is created with a low speed trephine under irrigation with 0.9% sterile saline. Final removal of the cranial piece is accomplished with a probe. Pre-weighed test article is then placed uniformly in the defect and soft tissues closed with interrupted resorbable suture. Care is taken not to perforate the dura and superior sagittal sinus. Animals are monitored throughout the 28-day test. Animals are euthanized using I.V. 0.5ml/300 gm Beuthanasia-D.

Craniotomy sites with 3 to 4 mm of surrounding bone are dissected from the fronto-occipital complex and immediately placed in 70% ethanol for further analysis.

After 24 hours in 70% ethanol, specimens are radiographed using high resolution radiographic film. Each roentgenogram is then digitized and radiopacity assessed within a standard 8 mm diameter circle superimposed over the defect site. The measured area of radiopacity within the standard circle is reported as a percentage of the total area.

After radiomorphometry, the specimens are further dehydrated in ethanol, embedded in methacrylate and microtomed in 4.5 μm coronal sections. Sections are prepared with trichrome stain for cellular detail and von Kossa stain for newly calcified tissue. Quantitative assessment of new bone formation within the defect site is assessed using von Kossa stained sections after a standard gray level is established between cellular structures and newly calcified tissue within the defect site. Descriptive statistics are performed on all test groups as part of the radiomorphometry and histomorphometry. Additional statistical analysis is accomplished by ANOVA with discreet comparisons evaluated by post-hoc testing and multiple comparisons using Fisher analysis.

Porcine bone matrix assay groups in this EXAMPLE include decalcified, irradiated bone particles as control and decalcified particles treated with ∞-galactosidase and each of buffer, guanidine hydrochloride or glutaraldehyde, terminally sterilized as enzyme/deantigenation test groups. The selection of test groups for this analysis includes three model control groups(a) an unfilled defect, and demineralized human matrix treated (b) with and (c) without guanidine hydrochloride to inactivate endogenous growth factors (Shigeyama et al., J. Periodontol. 66(6): 478-87 (1995)). The porcine test groups mirror the guanidine extraction for endogenous growth factor removal and include a non-enzymatically treated control. Previously developed deantigenation strategies have included aldehyde cross-linking and this processing variable is also included in a fourth porcine derived test group. The seven groups for this test are shown in TABLE VI below.

TABLE VI
Test Design for the Rat Cranial Defect Test

| | | |
|---|---|---|
| Group | | Number Of |
| Number | Test Group: Comment | Animals |

Porcine Bone Matrix

| | | |
|---|---|---|
| 1 | A decalcified, irradiated: control | 6 |
| 2 | B decalcified, ∝-gal, irradiated: active protein | 6 |
| 3 | C decalcified, ∝-gal, guanidine HCl, irradiated: inactivate | 6 |
| | protein | |
| 4 | D decalcified, ∝-gal, , no-irradiation: active protein | 6 |

Human Allograft Matrix and Controls

| | | |
|---|---|---|
| 5 | E decalcified, irradiated: active protein | 6 |
| 6 | F decalcified, guanidine HCl, irradiated: inactivate protein | 6 |
| 7 | G control defect: empty defect | 6 |

### EXAMPLE III

Z-bone Process

TABLE VII below provides steps for one embodiment of the Z-bone process:

TABLE VII

| | |
|---|---|
| 1. | Scrub frozen porcine thighs with disinfectant |
| 2. | Allow tissues to thaw |
| 3. | Remove soft tissue with boning knife |
| 4. | Scape remaining soft tissue with periosteal elevator |
| 5. | Remove proximal and distal metaphysis with oscillating saw |
| 6. | Cut bone shaft into manageable segments with oscillating saw |
| 7. | Ream out marrow with rotary reamer |
| 8. | Cut into small pieces and pool into basin with isopropanol |
| 9. | Transfer segments to vessel with hexane/methanol for 12-18 hours with constant |
| | agitation at 4° C. |
| 10. | Wash with WFI for 10-12 hours with constant agitation at 4° C., repeat 2 times. |
| 11. | Wash with WFI w/ 1.5M NaCl for 10-12 hours with agitation with lighting mixer (a310 |
| | impeller) at 4° C. |
| 12. | Inspect segments, remove any remaining soft tissue and transfer for new WFI bath for |
| | holding |
| 13. | Remove segments and reduce to appx. 2 cm pieces |
| 14. | Mill cold to <500 micron |
| 15. | Suspend resulting slurry in 70% IPA 0.1% Tween 20 and pour through stacked sieves |
| 16. | Pour three washes of 70% IPA Tween 20 through sieves |
| 17. | Collect 150-500 micron particles |
| 18. | Suspend bone in H₂O₂ and stir for 4-6 hours at 4° C. |
| 19. | Decant supernatant and add .5 N HCl (6 L) for 20-24 hours at 4° C. |
| 20. | 3 rinses with WFI |
| 21. | Decant supernatant and add α-galactosidase solution for 4-12 hours at 4-26° C. |
| 22. | Decant enzyme and perform three rinses with WFI |
| 23. | aliquot slurry into glass vial w/ stopper |
| 24. | Lyophilize 36-38 hours |
| 25. | Back fill vials with N₂ |
| 26. | Stopper and crimp and label vials |
| 27. | Irradiate with 2.0 mRad |
| 28. | Store at 4° C. or room temperature |

In another embodiment, the pilot process differs from TABLE VII above by one-step bulk lyophilizing with and dry particulate fill.

The details of one or more embodiments of the invention are set forth in the accompanying description above. Although any methods and materials similar or equivalent to those described herein can be used in the practice or testing of the invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

We claim:

1. An xenograft demineralized and deantigenated bone matrix, wherein the matrix has an increased osteoconductive and osteoinductive potential following treatment with ∞-galactosidase to eliminate ∞-Gal epitopes.

2. A method of using xenograft demineralized and deantigenated bone matrix as a bone graft, comprising:
implanting an improved xenograft demineralized and deantigenated bone matrix into the bone of a mammal.

3. The method of claim 2, wherein the mammal is a primate or a rodent.

4. The method of claim 3, wherein the primate is a human or rhesus monkey.

5. The method of claim 2, wherein the bone is cortical bone.

5. The method of claim 2, wherein the bone is cancellous bone.

5. The method of claim 2, wherein the graft is used for defects of the skeletal system.

6. The method of claim 2, wherein the efficacy of the graft is confirmed using a technique selected from the group consisting of quantitative radiography, histology, torsional biomechanics, and ectopic implantation testing.
